(19) United States
(12) Patent Application Publication
(10) Pub. No.: US 2005/0191205 A1
(43) Pub. Date: Sep. 1, 2005

(54) Indoor air quality module including a shield to minimize the leakage of ultraviolet light

(76) Inventors: Federico Uslenghi, Genova (IT); Francesco Antonione, Milano (IT); Marco Occhetta, Milano (IT)

Correspondence Address: CARLSON, GASKEY & OLDS, P.C., 400 WEST MAPLE ROAD, SUITE 350, BIRMINGHAM, MI, 48009, US

(21) Appl. No.: 10/789,699

(22) Filed: Feb. 27, 2004

(51) Int. Cl. A61L 9/00; A61L 2/00; A62B 7/08

(52) U.S. Cl. 422/24; 422/1; 422/4; 422/5; 422/121; 422/186.3; 422/306

(57) ABSTRACT

An indoor air quality module includes an ultraviolet light source located between two titanium dioxide coated honeycombs. When photons of ultraviolet light are absorbed by the titanium dioxide coating, reactive hydroxyl radicals are formed that attack and oxidize contaminants in the air to water, carbon dioxide, and other substances. A shield is positioned outside each of the honeycombs to minimize the direct leakage of ultraviolet light from the module. The height of each shield depends on the distance between each shield and the ultraviolet light source and the maximum angle that ultraviolet light can pass through the honeycomb without any reflection on the surface of the honeycomb. Any ultraviolet light that directly leaks from the honeycomb contacts the shield and reflects towards honeycomb, minimizing the direct leakage of ultraviolet light from the module and increasing the photocatalytic rate of the contaminants.

### BACKGROUND OF THE INVENTION

The present invention relates generally to an indoor air quality module including a reflective shield that minimizes direct leakage of ultraviolet light from the module and reflects the ultraviolet light towards a titanium dioxide coated honeycomb to increase the photocatalysis of contaminants in the air.

Indoor air can include trace amounts of contaminants, including biospecies, dust, particles, odors, carbon monoxide, ozone, and volatile organic compounds (VOCs) such as formaldehyde, acetaldehyde, toluene, propanol, butene, etc. Indoor air quality modules are used to purify the air by destroying contaminants. The module includes a titanium dioxide coated monolith, such as a honeycomb, and an ultraviolet light source.

Titanium dioxide operates as a photocatalyst to destroy contaminants when illuminated with ultraviolet light. Photons of the ultraviolet light are absorbed by the titanium dioxide, promoting an electron from the valence band to the conduction band, thus producing a hole in the valence band and adding an electron in the conduction band. The promoted electron reacts with oxygen, and the hole remaining in the valence band reacts with water, forming reactive hydroxyl radicals. When contaminants in the air flow through the honeycomb and are adsorbed onto the titanium dioxide coating, the hydroxyl radicals attack and oxidize the contaminants to water, carbon dioxide, and other substances. The ultraviolet light also kills the biospecies in the airflow that are irradiated.

If ultraviolet light leaks from the module, it may have several negative effects. For one, ultraviolet light may be harmful to the skin and to the eyes in high doses. Additionally, if ultraviolet-light leaks from the module, less ultraviolet light is directed to the titanium dioxide coating which reduces the number of hydroxyl radials and the photocatalytic effect of the titanium dioxide coating.

Hence, there is a need for an indoor air quality module that minimizes the direct leakage of ultraviolet light from the module and reflects the ultraviolet light towards the titanium dioxide coated honeycomb to increase the photocatalytic destruction of contaminants in the air.

### SUMMARY OF THE INVENTION

An indoor air quality module (IAQ) purifies the air in an interior space. The module includes an ultraviolet light source located between two titanium dioxide coated honeycombs. When photons of ultraviolet light are absorbed by the titanium dioxide coating, reactive hydroxyl radicals are formed. When contaminants such as a volatile organic compounds or carbon monoxide flow through the honeycomb and adsorb onto the titanium dioxide coating, the hydroxyl radicals attack the contaminants. A hydrogen atom is abstracted from the contaminants, oxidizing the contaminants to water, carbon dioxide, and other substances. The module also decomposes ozone to oxygen and kills biospecies.

The module includes a shield located outside each of the honeycombs to minimize direct leakage of ultraviolet light from the module. The height of the shields is less than the height of the honeycombs to allow air to flow above and below the shields. The height of each shield is related to the distance between the shield and the ultraviolet light source and the maximum angle that ultraviolet light can pass through the honeycomb with minimal reflection on the surface of the honeycomb.

Ultraviolet light that directly leaks from the honeycomb without reflection contacts the shield and reflects back towards the titanium dioxide coated honeycomb to minimize the direct leakage of ultraviolet light from the module and increasing the photocatalytic activity of the titanium dioxide coating.

Accordingly, the present invention provides an indoor air quality module that minimizes the direct leakage of ultraviolet light from the module and reflects the ultraviolet light towards the titanium dioxide coated honeycomb to increase the photocatalytic destruction of contaminants in the air.

### BRIEF DESCRIPTION OF THE DRAWINGS

The various features and advantages of the invention will become apparent to those skilled in the art from the following detailed description of the currently preferred embodiment. The drawings that accompany the detailed description can be briefly described as follows:

FIG. 1 schematically illustrates an enclosed environment, such as a building, vehicle or other structure, including an interior space and an HVAC system;

FIG. 2 schematically illustrates a side view of the indoor air quality module of the present invention including a shield that minimizes direct leakage of ultraviolet light from the module;

FIG. 3 schematically illustrates a front view of a honeycomb;

FIG. 4 schematically illustrates a side view of the honeycomb and the travel of ultraviolet light through the honeycomb and reflected by the honeycomb;

FIG. 5 schematically illustrates a front view of the module of FIG. 2 taken along line 5-5; and

FIG. 6 schematically illustrates a side view of the distances between the honeycomb, the ultraviolet light source, and the shields.

### DETAILED DESCRIPTION OF THE PREFERRED EMBODIMENT

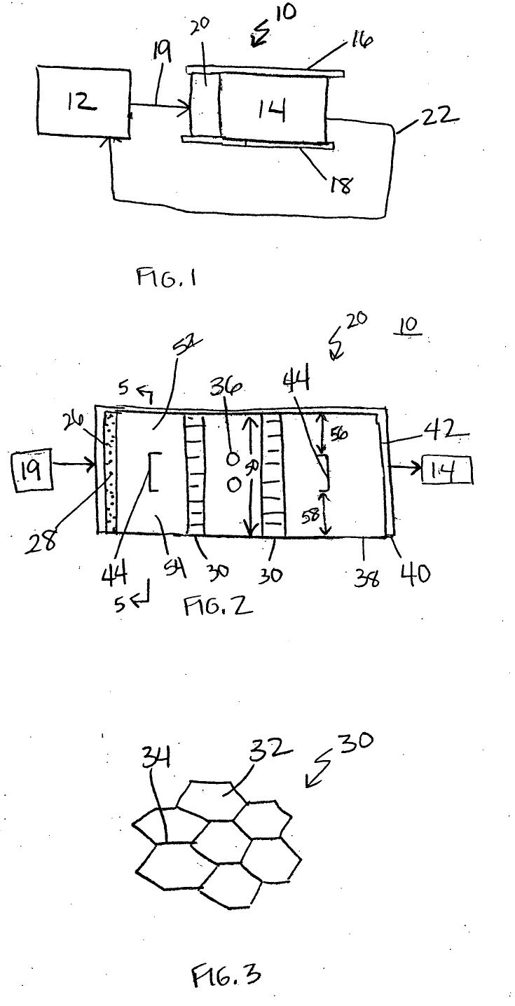

FIG. 1 schematically illustrates a structure 10, such as building or vehicle, that includes an interior space 12. The interior space 12 can be a room, an office or a vehicle cabin, such as a car, train, bus or aircraft. An HVAC system, such as a satellite indoor unit 14, heats or cools the interior space 12 of the structure 10. The satellite indoor unit 14 preferably is installed between a ceiling 16 and a false ceiling 18 in the structure 10. It should be understood that other arrangements will benefit from this invention.

Air in the interior space 12 is drawn into the satellite indoor unit 14 through an air duct 19. The satellite indoor unit 14 changes the temperature of the air drawn into the air duct 19. If the satellite indoor unit 14 is operating in a cooling mode, the air is cooled. Alternately, if the satellite indoor unit 14 is operating in a heating mode, the air is heated. The air is then returned to the interior space 12 through an air duct 22 to change the temperature of the air in the interior space 12.

An indoor air quality module 20 mounted between the air duct 19 and the satellite indoor unit 14 purifies the air before it is drawn into the satellite indoor unit 14. Alternately, the module 20 can purify the air leaving the satellite indoor unit 14 before returning into the interior space 12 or the module 20 can be a stand alone unit employed without the satellite indoor unit 14.

The indoor air quality module 20 oxidizes contaminants in the air, including volatile organic compounds, semi-volatile organic compounds and carbon monoxide, to water, carbon dioxide, and other substances. Examples of volatile organic compounds are aldehydes, ketones, alcohols, aromatics, alkenes, or alkanes. The indoor air quality module 20 also decomposes ozone to oxygen and kills biospecies.

FIG. 2 schematically illustrates a side view of the indoor air quality module 20 of the present invention. The indoor air quality module 20 defines a compartment. The air flows through a particle filter 28 that filters dust or other large particles from the air.

The filtered air then flows through a monolith 30, such as a honeycomb 30 (FIG. 3). Preferably, there are at least two honeycombs 30 in the module 20 made of aluminum or an aluminum alloy. FIG. 3 schematically illustrates a front view of a portion of a honeycomb 30. The honeycomb 30 includes a plurality of hexagonal open passages 32 through which the air flows. The open passages 32 are coated with a photocatalytic coating 34, such as titanium dioxide. The titanium dioxide can also be doped or loaded with a metal oxide.

An ultraviolet light source 36 is positioned between the honeycombs 30. The ultraviolet light source 36 generates light having a wavelength in the range of 180 to 400 nanometers. If more than two honeycombs 30 are utilized in the module 20, the honeycombs 30 and the ultraviolet light source 36 alternate in the indoor air quality module 20. That is, an ultraviolet light source 36 is located between each of the honeycombs 30.

When illuminated by the ultraviolet light source 36, the titanium dioxide coating 34 on the honeycomb 30 is activated. Photons of ultraviolet light are absorbed by the titanium dioxide coating 34, promoting an electron from the valence band to the conduction band and producing a hole in the valence band. The electrons promoted to the conduction band are captured by oxygen. The holes in the valence band react with water molecules adsorbed on the titanium dioxide coating 34 to form reactive hydroxyl radicals.

When a volatile organic compound adsorbs onto the titanium dioxide coating 34, the hydroxyl radicals attack the volatile organic compound, abstracting a hydrogen atom from the volatile organic compound. The hydroxyl radicals oxidize the volatile organic compounds and produce water, carbon dioxide, and other substances. The purified air then exits the indoor air quality module 20 through an outlet 42.

As air flow through the module 20, the particle filter 28 acts as a mechanical filter to remove dust and particles. When illuminated by the ultraviolet light source 36, the titanium dioxide coated 34 honeycombs 30 oxidize and destroy volatile organic compounds. Finally, the ultraviolet light generated by the ultraviolet light source 36 has a germicidal effect to kill biospecies.

The indoor air quality module 20 further includes an inner compartment 38 and an outer compartment 40. The particle filter 28, the honeycombs 30 and the ultraviolet light source 36 are contained in the inner compartment 38. The outer compartment 40 is attached to the air duct 19 and to the satellite indoor unit 14 and houses the electric, electronic and safety related components. During operation of the module 20, the inner compartment 38 is contained in the outer compartment 40. When servicing is required, the inner compartment 38 is detached from the outer compartment 40 to allow access to and repair of the components in the inner compartment 38.

The indoor air quality module 20 further includes a shield 44 positioned outside each honeycomb 30. That is, the ultraviolet light source 36 is located on one side of each honeycomb 30 and a shield 44 is located on the opposing side of each honeycomb 30. The shields 44 minimize direct leakage of ultraviolet light from the module 20. Preferably, the shields 44 are made of sheet metal. When installed, the shields 44, the honeycombs 30 and the ultraviolet light source 36 are all parallel in the inner compartment 38. The shields 44 and the honeycombs 30 also have the same width.

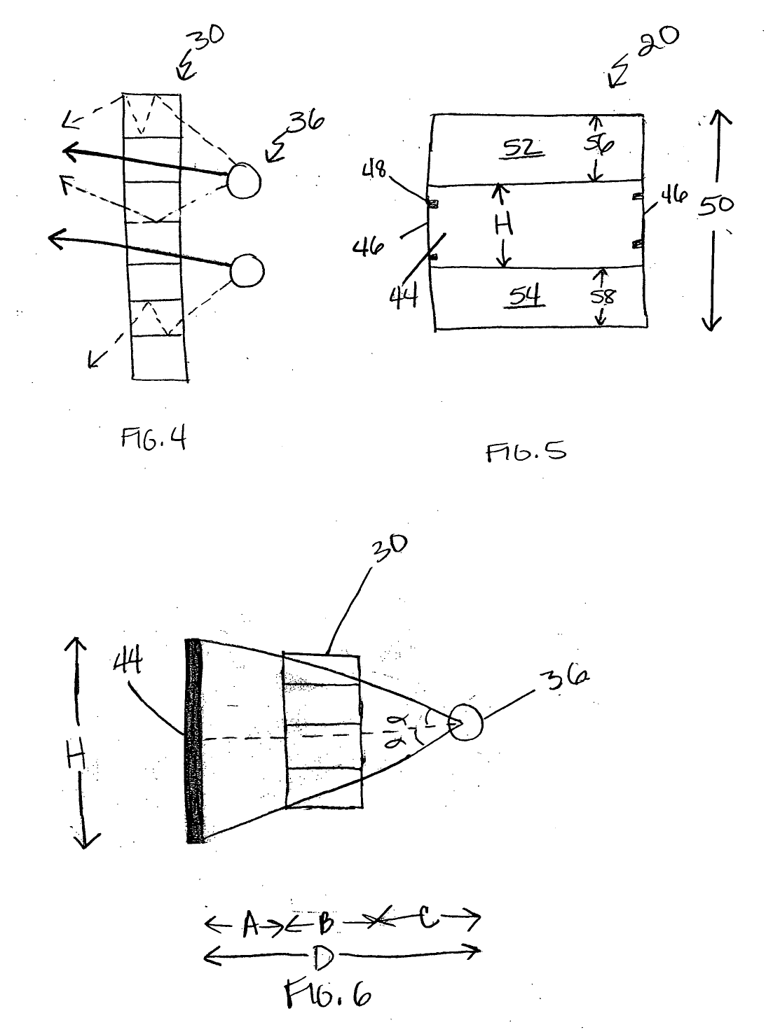

Referring to FIG. 4, direct leakage (solid lines) of the ultraviolet light is defined herein as any ultraviolet light that passes through the honeycomb 30 without reflection off the surface of the honeycomb 30. The solid arrows represent the direct leakage of the ultraviolet light through the honeycomb 30. Indirect leakage (phantom lines) of the ultraviolet light is defined as any ultraviolet light that passes through the honeycomb 30 with at least one reflection off the surface of the surface of the honeycomb 30. The phantom arrows represent the indirect leakage of the ultraviolet light through the honeycomb 30. As ultraviolet light contacts the honeycomb 30, the intensity and the potential harmful effects of the ultraviolet light is decreased.

Referring to FIG. 5, the opposing sides 46 of the shield 44 are attached to the inner compartment 38 of the module 20 by a fastener 48, such as a threaded fastener. In one example, the threaded fastener is a screw. The shield 44 has a height H, and the height H of the shield 44 is less than the height 50 of the inner compartment 38. When the shield 44 is installed in the inner compartment 38, openings 52 and 54 are defined above and below the shield 44, respectively, to allow for the passage of air. When the shield 44 is installed in the inner compartment 38, the height 56 of the opening 52 is substantially equal to the height 58 of the opening 54. That is, the shield 44 is centered in the inner compartment 38.

As illustrated in FIG. 6, the height H of the shield 44 is selected to minimize or minimize the direct leakage of ultraviolet light from the module 20. The height H of the shield 44 is determined by the following equation:
H=2*D*tan(α)

The variable D is defined as the distance D from the shield 44 to the center of the ultraviolet light source 36. The distance D equals the distance A from the shield 44 to the honeycomb 30, plus the thickness B of the honeycomb 30, plus the distance C from the honeycomb 30 to the center of the ultraviolet light source 36. The angle alpha (α) is defined as the maximum angle from the horizontal that the ultraviolet light can pass through the honeycomb 30 without any reflection on the surface of the honeycomb 30. At angles greater than α, the ultraviolet light will contact and reflect on the surface of the honeycomb 30. By determining the angle α and the distance D and using the above equation, the height H of the shield 44 can be calculated. The shield 44 then has a height H great enough to minimize direct leakage of ultraviolet light from the module 20 but not oversized so as to minimize the flow of air through the openings 52 and 54 above and below the shield 44, respectively.

When the shield 44 is installed in the inner compartment 44, the shield 44 and the ultraviolet light source 36 must be parallel to maintain the distance D as a constant. Additionally, the ultraviolet light source 36 is aligned with the center of the shield 44. That is, the distances from the ultraviolet light source 36 to each of the ends of the shield 44 are equal.

When the shield 44 is installed in the inner compartment 38, ultraviolet light that directly leaks from the honeycomb 30 will contact the shield 44 and reflect back towards honeycomb 30, minimizing the direct leakage of ultraviolet light from the module 20. As more ultraviolet light is directed towards the honeycomb 30, the photocatalytic rate of the contaminants in the air increases, reducing the amount of contaminants in the air and the overall effectiveness of the module 20.

The foregoing description is only exemplary of the principles of the invention. Many modifications and variations of the present invention are possible in light of the above teachings. The preferred embodiments of this invention have been disclosed, however, so that one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

1. An indoor air quality module comprising:
a compartment having an inlet and an outlet;
a monolith located between the inlet and the outlet;
a photocatalytic coating on the monolith;
an ultraviolet light source which directs ultraviolet light towards the photocatalytic coating; and
a shield adjacent the monolith.

2. The module as recited in claim 1 wherein the shield reflects the ultraviolet light that passes through the monolith towards the monolith to minimize leakage of the ultraviolet light from the module.

3. The module as recited in claim 1 wherein the photocatalytic coating is titanium dioxide.

4. The module as recited in claim 1 wherein the monolith comprises a honeycomb.

5. The module as recited in claim 4 wherein the honeycomb comprises a plurality of hexagonal shaped passages coated with the photocatalytic coating.

6. The module as recited in claim 1 wherein the shield comprises a sheet metal.

7. The module as recited in claim 1 wherein the shield has an upper edge and an opposing lower edge.

8. The module as recited in claim 7 wherein an upper gap is defined between the upper edge and the compartment and a lower gap is defined between the opposing lower edge and the compartment.

9. The module as recited in claim 8 wherein the upper gap has an upper gap height and the lower gap has a lower gap height, and the upper gap height is substantially equal to the lower gap height.

10. The module as recited in claim 1 wherein the monolith comprises a first monolith and a second monolith, the ultraviolet light source located between the first monolith and the second monolith.

11. The module as recited in claim 1 wherein the monolith defines a monolith height and the shield defines a shield height, the shield height is less than the monolith height.

12. The module as recited in claim 11 wherein a distance is defined between the ultraviolet light source and the shield and a non-reflection angle is defined as a maximum angle from a horizontal that the ultraviolet light can pass through the monolith without contacting the monolith, and wherein the shield height relates to the distance and the non-reflection angle.

13. The module as recited in claim 1 wherein the shield height is defined by a variable H, the distance is defined by the variable D, and the non-reflection angle is defined by the variable α, and the shield height is determined by the following equation:
H=2*D*tan(α)

14. An indoor air quality module comprising:
a compartment having an inlet and an outlet;
a first monolith located between the inlet and the outlet of the compartment and having a monolith height;
a second monolith located between the inlet and the outlet of the compartment and having the monolith height;
a photocatalytic coating on the first monolith and the second monolith;
an ultraviolet light source adjacent the first monolith and the second monolith which directs ultraviolet light towards the photocatalytic coating; and
a first shield having a shield height less than the monolith height; and
a second shield having the shield height, the first monolith and the second monolith located between the first shield and the second shield, and the first shield reflects the ultraviolet light that passes through the first monolith towards the first monolith to minimize leakage of the ultraviolet light from the module and the second shield reflects the ultraviolet light that passes through the second monolith towards the second monolith to minimize leakage of the ultraviolet light from the module.

15. The module as recited in claim 14 wherein the shield comprises an upper edge and an opposing lower edge, and an upper gap is defined between the upper edge and the compartment and a lower gap is defined between the opposing lower edge and the compartment, and wherein the upper gap has an upper gap height and the lower gap has a lower gap height, and the upper gap height is substantially equal to the lower gap height.

16. The module as recited in claim 14 wherein a first distance is defined between the ultraviolet light source and the first shield and a second distance is defined between the ultraviolet light source and the second shield, and a non-reflection angle is defined as a maximum angle from a horizontal that the ultraviolet light can pass through the first monolith and the second monolith without contacting the first monolith and the second monolith, and wherein the shield height of the first shield depends on the first distance and the non-reflection angle and the shield height of the second shield depends on the second distance and the non-reflection angle.

17. A method of purifying air comprising the steps of:
(a) flowing the air through a monolith having a photocatalytic coating;
(b) illuminating the photocatalytic coating on the monolith with ultraviolet light; and
(c) reflecting the ultraviolet light that passes through the monolith towards the monolith to minimize leakage of the ultraviolet light.

18. The method as recited in claim 17 wherein said step (b) produces hydroxyl radicals to destroy contaminants in the air and destroying the contaminants with the hydroxyl radicals.

19. The method as recited in claim 17 wherein an ultraviolet light source illuminates the photocatalytic coating and a shield reflects the ultraviolet light, and the method further comprises the steps of defining a distance between the ultraviolet light source and the shield and defining a non-reflection angle as a maximum angle from a horizontal that the ultraviolet light can pass through the monolith without contacting the monolith, and wherein a height of the shield relates to the distance and the non-reflection angle.

20. The method as recited in claim 17 wherein the ultraviolet light that passes through the monolith without reflection is defined as direct leakage.
